(19) United States
(12) Patent Application Publication
(10) Pub. No.: US 2003/0176406 A1
(43) Pub. Date: Sep. 18, 2003

(54) Polycaprolactone-b-poly (ethylene oxide) copolymer non-cross-linked micelles as a delivery vehicle for steroid

(75) Inventors: Christine Allen, Richmond (CA); Adi Eisenberg, Montreal (CA); Dusica Maysinger, Montreal (CA)

Correspondence Address: SWABEY OGILVY RENAULT, Suite 1600, 1981 McGill College Ave., Montreal, QC, H3A 2Y3, CA

(73) Assignee: McGill University

(21) Appl. No.: 10/314,988

(22) Filed: Dec. 10, 2002

Related U.S. Application Data
(63) Continuation-in-part of application No. 09/577,936, filed on May 25, 2000, now abandoned.
(60) Provisional application No. 60/136,757, filed on May 28, 1999.

(51) Int. Cl. A61K 31/56; A61K 9/14

(52) U.S. Cl. 514/177; 424/486

(57) ABSTRACT

The present invention relates to non-cross-linked micelles as delivery vehicles for steroid compounds and more particularly to a polycaprolactone-b-poly(ethylene oxide) copolymer micelle as a delivery vehicle for steroids. The delivery system comprises a population of diblock copolymer non-cross-linked micelles, each micelle defining a non-cross-linked core for containing the steroid compound. The delivery system of the present invention maintains the release of the steroid compound in a patient having a deficient steroid level.

### RELATED APPLICATION

This application is a continuation-in-part of application Ser. No. 09/577,936 filed on May 25, 2000, which is still pending and which is hereby incorporated by reference.

### BACKGROUND OF THE INVENTION

(a) Field of the Invention

The present invention relates to non-cross-linked micelles as delivery vehicles and more particularly to polycaprolactone-b-poly(ethylene oxide) copolymer non-cross-linked micelles as delivery vehicles for steroids.

(b) Description of Prior Art

In recent years, there has been much interest in the use of androgen replacement therapy for treating a variety of clinical indications. Currently, the most common use is for the treatment of men with hypogonadism, in which case testosterone production falls below a normal range of 3-10 mg/day. Hypogonadism is characterized by a loss of muscle and bone mass, an increase in visceral fat, impaired immune function and altered mood. Other accepted indications requiring androgen replacement therapy are microphallus in infants and delayed puberty in boys. Presently, androgen therapy is under investigation for the treatment of aging men with low to normal testosterone levels. Serum testosterone levels have been shown to decline with aging in men. This decline is gradual in comparison to the decline in production of oestrogen in postmenopausal women and there is much inter-individual variation. The issue of androgen supplementation in aging men requires further investigation in order to ensure that the benefits do outweigh the risk posed to the cardiovascular system and the prostate. However, results from studies on aging men given androgen replacement therapy have been quite promising. Androgen replacement therapy is also being considered for the treatment of postmenopausal women and individuals in wasting states owing to their affliction with HIV, cancer or chronic infection.

The increasing interest in androgen replacement therapy has prompted the development of many androgen preparations, including patches, creams, gels, injectables and implants. Many of the oral androgen formulations have been found to have low potency and may be either rapidly cleared by the liver or potentially hepatotoxic. At present, the most commonly used androgen preparations worldwide are testosterone enanthate and cypionate injections. However, the pharmacokinetic profile of testosterone administered in these preparations is somewhat undesirable since the serum testosterone level is characterized by peaks and troughs within the 14 days following injection: peak serum testosterone levels are reached following 1-3 days and then fall below physiological level between days 10-14. This type of profile does not mimic the physiologic cycle for testosterone and gives rise to undesirable side effects such as skin problems (e.g. acne, oiliness) during the peak periods and mood changes and loss of libido when testosterone levels fall below physiological levels.

Biodegradable microspheres containing testosterone have been used for androgen replacement therapy. The administration of the microsphere preparation to hypogonadal men gave rise to physiological serum testosterone levels for 10-11 weeks following a single injection. Several other colloidal carriers have been tried as drug delivery vehicles for steroids (Hagan S. A et al., Langmuir, 12 (1996)2153-2161, which is hereby incorporated by reference along with any other scientific references referred below) These include microspheres composed of homopolymer (e.g. poly (d,l-lactic acid)) and copolymer (e.g. poly(lactide)-co-poly(e-caprolactone)) materials. Also, micelles formed from copolymers of poly(lactide)-b-poly(ethylene glycol) were studied as a carrier for testosterone (Hagan S. A et al., Langmuir, 12 (1996)2153-2161).

The characterization of the PCL₂₀-b-PEO₄₄ micelles has been described (Allen C., Yu Y., Maysinger D., Eisenberg A., Bioconjugate Chem. 9 (1998) 564-572). Block copolymer micelles have been investigated as delivery vehicles for lipophilic compounds (Hagan S. A et al., Langmuir, 12 (1996)2153-2161). The amphiphilic nature of the copolymer molecules enables them to self-assemble to form micelles in an aqueous medium. The micelle is composed of a hydrophobic core that serves as cargo space for the lipophilic drugs and a hydrophilic corona that acts as an interface between the core and the external medium.

There is described in U.S. Pat. No. 5,429,826 in the name of Nair, a oleophilic cross-linked core which would not be expected to house a lipophilic compound as opposed to the non-cross-linked polymer of the present invention. Further the Nair patent does not teach that cross-linked polymer can actually “house” a lipophilic compound.

From small molecule literature, non-cross-linked micelles are expected to dissolve because of the high critical micelle concentration of small molecule micelles (See Table 16.2, Intermolecular and Surface Forces, Jacob Israelachvilli, Academic Press, 1992, pg 355-357).

It would therefore be highly desirable to be provided with a delivery system for a sustained release and a prolonged delivery of a steroid compound in a patient, in an amount without the side effects that such an amount delivered with the systems of the prior art would cause.

### SUMMARY OF THE INVENTION

One aim of the present invention is to provide a delivery system providing sustained delivery of a steroid compound in a patient, in an amount without the side effects that such an amount delivered with the systems of the prior art causes.

In accordance with the present invention, there is provided a delivery system for a maintained delivery of a steroid compound to a patient having a deficient steroid level. The delivery system comprises a population of diblock copolymer non-cross-linked micelles, each micelle defining a core for containing the steroid compound, to increase the steroid level in the patient to a physiologically acceptable level.

The diblock copolymer may consist of polycaprolactone-b-poly(ethylene oxide).

The polycaprolactone-b-poly(ethylene oxide) copolymer may comprise a number of caprolactone monomers selected from 5 to 150 and a number of ethylene oxide monomers selected from 20 to 100.

The selected number of caprolactone monomers may consist of 20 and the selected number of ethylene oxide monomers may consist of 44.

The steroid compound may consist of a steroid hormone.

The steroid hormone may consist of an androgen.

The androgen may consist of dihydrotestosterone.

In accordance with the present invention, there is also provided a method for treating a patient having a steroid level deficiency. The method comprises administering a population of diblock copolymer non-cross-linked micelles containing a steroid compound to the patient, the micelles increasing the steroid level to a physiologically acceptable level and maintaining the level.

The diblock copolymer may consist of polycaprolactone-b-poly(ethylene oxide).

The steroid level deficiency may be associated but is not limited to conditions selected from a group consisting of hypogonadism, microphallus, delayed puberty, post-menopause, HIV, cancer and chronic infections.

The term “steroid” is intended to mean a steroid hormone or a steroid compound that increases the level of an endogenous steroid hormone in a patient.

The micelles of the present invention are non-cross-linked micelles or micelles devoid of a cross-linked core, as a result, effectively and surprisingly capable of housing an amount of a steroid compound and provide a sustained delivery of a physiologically acceptable level of such a steroid compound to a patient. The release kinetics of the micelles of the present invention are controllable and particularly favor the release of steroids contained therein to ultimately treat a condition associated with a steroid deficiency.

A preferred delivery system includes, without limitation, PCL₂₀-b-PEO₄₄ (where subscripts refer to block lengths) copolymer non-cross-linked micelles as a carrier for androgens.

The delivery system of the present invention provides a pharmacokinetic profile for the androgens released with such a delivery system that provides controlled release.

Block copolymer non-cross-linked micelles formed from copolymers of poly(caprolactone)-b-poly(ethylene oxide) (PCL-b-PEO) were investigated as a drug delivery system for dihydrotestosterone (DHT). The physical parameters of the PCL-b-PEO micelle-incorporated DHT were measured, including the loading capacity of the micelles for DHT, the relative ratio (Kᵣ) of DHT that partitions between the micelles and the external medium and the kinetics of the release of DHT from the non-cross-linked micelle solution. The MTT survival assay was used to assess the in vitro biocompatibility of PCL-b-PEO micelles in HeLa cell cultures. The biological activity of the non-cross-linked micelle-incorporated DHT was evaluated in HeLa cells co-transfected with the expression vectors for the androgen receptor and the MMTV-LUC reporter gene. The PCL-b-PEO micelles were found to have a high loading capacity for DHT and the release profile of the drug from the micelle solution was found to be a slow steady release that continued over a one month period. The in vitro biocompatibility of the micelles was confirmed in HeLa cell cultures and the biological activity of the non-cross-linked micelle-incorporated DHT was fully retained.

More particularly, the physico-chemical characterization and in vitro study of PCL₂₀-b-PEO₄₄ non-cross-linked micelles as a delivery vehicle for DHT are herein disclosed. The loading capacity of the non-cross-linked micelles is determined and several parameters are evaluated, including the number of DHT molecules per micelle, the Kᵣ of DHT that partitions between the micelle and the external medium and the release profile of the non-cross-linked micelle-incorporated DHT. In addition, the in vitro cytotoxicity of a range of concentrations of the PCL₂₀-b-PEO₄₄ copolymer micelles are studied in HeLa cells and evaluated using the MTT survival assay. The biological activity of the non-cross-linked micelle-incorporated DHT is then evaluated in HeLa cells co-transfected with the MMTV-LUC reporter gene and the androgen receptor.

### BRIEF DESCRIPTION OF THE DRAWINGS

FIG. 1 illustrates the plot of the Kᵣ of DHT (left axis) which partitions between the PCL₂₀-b-PEO₄₄ micelles and the external medium versus the amount of DHT added during micelle preparation and the amount of DHT loaded into the PCL₂₀-b-PEO₄₄ micelles (right axis) versus the amount of DHT added during non-cross-linked micelle preparation;

FIG. 2 illustrates the plot of (right axis) the number of molecules of DHT loaded per micelle (actual=full line, maximum if 100% DHT was incorporated=dashed line versus the amount of DHT added during micelle preparation and the ratio of the weight of drug loaded to the total weight of micelle cores (left axis) versus the amount of drug added during micelle preparation;

FIG. 3 illustrates the release of DHT alone (X), of 8 mM micelle-incorporated DHT (□) and of 40 mM micelle-incorporated DHT (σ) over time;

FIG. 4 illustrates the in vitro biocompatibility of PCL₂₀-b-PEO₄₄ micelles in HeLa cells for 24, 48 and 72 hours as measured relative to the untreated control which is taken to be 100% survival; and

FIG. 5 illustrates the luciferase activity in HeLa cells cotransfected with MMTV-LUC and the androgen receptor and treated with either nothing (control), 100 nM of DHT alone, empty PCL₂₀-b-PEO₄₄ micelles or 100 nM of PCL₂₀-b-PEO₄₄ micelles-incorporated DHT.

### DETAILED DESCRIPTION OF THE INVENTION

The PCL₂₀-b-PEO₄₄ block copolymer was synthesized by anionic polymerization. All chemicals were purchased from Aldrich Chemical Company. The scintillation cocktail used in loading studies (Fisher Chemical, Fisher Scientific, ScintiSafe Econo F) and that used in the kinetic release studies (Fisher Chemical, Fisher Scientific, ScintiSafe™ Plus 50%) was purchased from V.W.R. Scientific. The radiolabeled DHT was purchased from Mandel Scientific while the cold dihydrotestosterone was purchased from Sigma. The dialysis bag used in the non-cross-linked micelle preparation was Spectral Por™ Membrane, MWCO=50,000 and that used for the kinetic release studies was also Spectral Por™ Membrane but MWCO=15,000.

Preparation of Dihydrotestosterone Incorporated PCL₂₀-b-PEO₄₄ Non-Cross-Linked Micelles

To a small glass vial, 0.005 g of the PCL₂₀-b-PEO₄₄ copolymer and an aliquot of a DHT stock solution containing a known ratio of radiolabeled to non-radiolabeled drug were dissolved in 0.15 g of dimethylformamide (DMF). This solution was stirred using a magnetic stir bar for 4 hours. To induce non-cross-linked micelle formation, a 0.35 g of double distilled water was added slowly with stirring (one drop every ten seconds) to the copolymer-drug solution. The total mass of this mixture was 0.5 g, which yielded a 1% polymer solution by weight. This solution was stirred for 12 hours. To remove the DMF and the excess DHT, the solution was dialyzed in double distilled water for 12 hours. The water was changed twice every 4 hours and then once every hour for 4 hours. After dialysis, the non-cross-linked micelle solution was available for use.

Measurement of the Amount of DHT Loaded in PCL₂₀-b-PEO₄₄ Micelles

An aliquot of the non-cross-linked micelle solution was dried in a vacuum dessicator in a small glass vial. The micelles were then dissolved in 450 μL of DMF, stirred by vortex and then counted using 4 mL of scintillation cocktail (Wallac Liquid Scintillation Counter). The number of counts per minute for 100 μL of micelle solution was used to extrapolate the number of counts for a 500 μL sample. The counts per minute for each solution were then converted to moles using a calibration curve.

Kinetic Release of DHT from PCL₂₀-b-PEO₄₄ Micelles

The release kinetics of DHT from PCL₂₀-b-PEO₄₄ non-cross-linked micelles was performed using previously dried 100 μL aliquots of micelle solution. To the dried micelles, 100 μL of phosphate buffer solution, PBS (pH 7.4) was added. To ensure that the micelles were well dispersed in PBS, the solutions were vortexed. The dispersed micelles were left to stand in a 37° C. warm room for 30 minutes. Once the micelle solution had reached 37° C., it was placed in a dialysis bag which was immersed in a large vial containing warmed PBS (10 mL PBS, pH=7.4, 37° C.). At the appropriate time intervals, a 100 μL aliquot of PBS was removed from the large vial to count the amount of DHT released from the micelles. The aliquot was dispersed in 4 mL of scintillation cocktail and then counted. Following the removal of each 100 uL aliquot, a 100 uL aliquot of fresh PBS was added.

Relative Ratio of DHT Incorporated PCL₂₀-b-PEO₄₄ Micelles

Immediately after the dialysis of non-cross-linked micelles, 100 μL aliquots of solution were placed into Eppendorf vials. The solutions were then centrifuged at 14,000 rpm for 10 minutes (Eppendorf Centrifuge 5402). The supernatant was removed carefully by pipette and placed into a glass vial containing 450 μL of DMF. The precipitate (micelles) was left in the Eppendorf and to it, 450 μL of DMF was added. These solutions were stirred using the vortex for several minutes. Each of the solutions were then added to vials containing 4 mL of scintillation cocktail and counted with the scintillation counter.

In Vitro Experiments

Cell Culture

HeLa cells (ATCC) were grown in 6 well plates (Falcon) in DMEM medium supplemented with penicillin (1%) and streptomycin (1%) and 10% fetal bovine serum to reach 60% confluency.

In Vitro Cytotoxicity of PCL₂₀-b-PEO₄₄ Non-Cross-Linked Micelles

The cells were split into 24 well plates (500 μL medium per well) for the cytotoxicity studies. An aliquot of a stock solution of PCL₂₀-b-PEO₄₄ micelles was added to each well such that the concentration of empty micelles ranged from 1×10⁻⁶ to 1×10⁻³ (g/g) per well. Following 24, 48 or 72 hours the cell survival was measured using the MTT assay. The MTT dye was 10 added to each well following the specific time period and incubated for 4 hours at 37° C. The cells were then placed on ice and the supernatant was removed by aspiration. An aliquot of trypsin was then added to each well and the cells were left on ice for 20 minutes. DMSO was then added to each well and the cells were collected and centrifuged at 14,000 rpm for 20 minutes at 4° C. The supernatants were then collected and the absorbance of each was measured at λ=595 nm.

Cell Transfection

The medium was changed 4 hours prior to the transfection procedure. HeLa cells were transfected by applying the calcium phosphate transfection protocol. The cells were transfected with expression vectors encoding the androgen receptor and the reporter plasmid MMTV-LUC (kindly provided by Dr. Albert O. Brinkmann, Erasmus University, Rotterdam). The full description of the AR construct is described elsewhere. Following a 48-hour period, the medium was removed and replaced by androgen free medium (serum treated with charcoal to eliminate steroids) and the cells were treated as described below.

Cell Treatment

To assess the effects of dihydrotestosterone and non-cross-linked micelle-incorporated dihydrotestosterone on the induction of the integrated MMTV-LUC gene, the cells were treated as follows: no treatment (negative control), empty micelles (20 uL of a 1% (w/w) solution, negative control), DHT alone (positive control) or micelle-incorporated DHT, such that the final concentration of DHT in each well was 100 nM. Each condition was assessed in triplicate per experiment and three separate experiments were carried out (n=9) The cells were allowed to incubate with treatment for 24 hours prior to performing the luciferase assay.

Assay for Luciferase Activity in Transfected HeLa Cells

The treated or non-treated transfected HeLa cells were lysed in a standard lysis buffer: 25 mM triphosphate, pH 7.8; 8 mM magnesium chloride; 1 mM DTT; 1 mM EDTA; 1% Triton X-100; 1% BSA and 15% glycerol. Upon cell lysis, the cells were scraped with a rubber policeman and the lysates were collected in Eppendorf tubes and stored at −80° C. The lysates were then thawed and centrifuged for 15 minutes at 13,000 rpm and 4° C. The luciferase assay was performed using the Promega luciferase assay system and all steps were performed on ice. A 100 uL aliquot of the luciferase assay reagent was then added to 100 μL of the extract in an Eppendorf tube. An aliquot of the luciferase assay substrate was added to the sample, mixed and the sample was introduced into the counting chamber. A constant time interval was maintained between substrate addition and data acquisition (10-15 secs). The data acquisition was carried out over 2 minutes.

Light Intensity Measurements

The light intensity was measured using a LKB model 1211 Rackbeta liquid scintillation counter.

Loading Capacity of PCL₂₀-b-PEO₄₄ Micelles for Dihydrotestosterone

In the final stage of non-cross-linked micelle preparation, the micelles are dialyzed against double distilled water in order to remove the organic solvent (DMF). During this stage, both the drug that was not incorporated into the micelles and some of the drug that was incorporated are lost and go to waste.

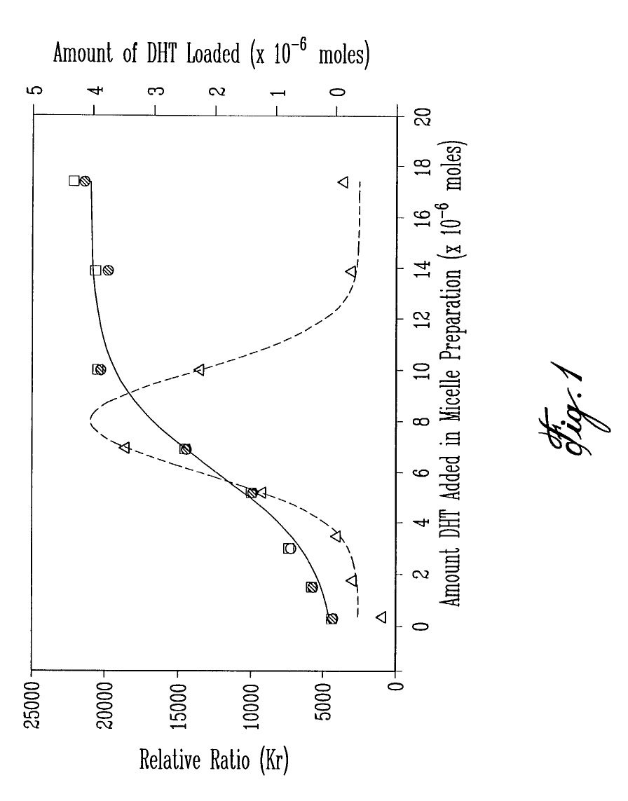

FIG. 1 illustrates the plot of the amount of DHT loaded in the micelle solution versus the total amount of DHT added (0.3-17.4 μmoles) during the preparation of 0.1 mL of a 1% (w/w) solution of PCL₂₀-b-PEO₄₄ micelles. The amount of DHT incorporated into the 0.1 mL volume of the micelle solution increases as the amount of DHT added during micelle preparation is increased until a maximum loading capacity is reached. The maximum amount of DHT that may be loaded into the 0.1 mL volume was found to be 4.4 μmoles, which corresponds to 1.3 mg of DHT. This maximum loading capacity is reached when 10 μmoles or more of DHT are added during micelle preparation. In this way, the loading efficiency, meaning the percentage of the total drug added during micelle preparation that is loaded into the micelle solution, decreases from 39% when 10 μmoles DHT are added during preparation to 23% when 17 μmoles are added.

Relative Ratio of Micelle-Incorporated DHT to DHT in the External Medium of the Micelle Solution

For the drug that has been incorporated into the micelle solution, an equilibrium exists whereby the drug partitions between the micelle cores and the external medium. The partition coefficient (Kᵥ) for drugs or model hydrophobic compounds (pyrene) between the micelles and the external medium has been described previously in detail. In this case, since the measurements of the amount of drug in the micelles and the external medium are carried out following dialysis this is termed the relative ratio (Kᵣ) rather than the Kᵥ since the process has been influenced by the dialysis process. The Kᵣ may be described by the following equation:

$$\frac{{[ DHT ]}_{micelles}}{{[ DHT ]}_{water}} = \frac{Kr {χ}_{\underline{PCL}} C}{{ρ}_{CL}}$$

where [DHT]_{micelles} is the concentration of DHT in the micelles (precipitate following centrifugation), [DHT]_{water} is the concentration of DHT in the external medium (supernatant following centrifugation), χ_{PCL} is the weight fraction of PCL in the PCL₂₀-b-PEO₄₄ copolymer (χ_{PCL}=0.55), C is the concentration of copolymer in g/mL and ρ_{CL} is the density of PCL in g/mL (ρ_{CL}≅1).

FIG. 1 includes the plot of the relative ratio (Kᵣ) versus the amount of DHT added during micelle preparation. The value of Kᵣ is found to range from a minimum of 833 to a maximum of 18,500. The value of Kᵣ increases as the amount of DHT during micelle preparation increases from 0.35-6.94×10⁻⁶ moles, at which point it reaches a maximum value of 18,500. Beyond this point the relative ratio decreases as more of the DHT is found in the external medium.

Number of Molecules Loaded per Micelle

From the known amount of DHT incorporated into the non-cross-linked micelles, it is possible to calculate the number of molecules incorporated per micelle. The number of micelles present in solution may be calculated if the aggregation number (N_{A}) for the copolymer is known. The N_{A} is the number of copolymer molecules that aggregate to form a micelle. The N_{A} for PCL₂₀-b-PEO₄₄ is 125.

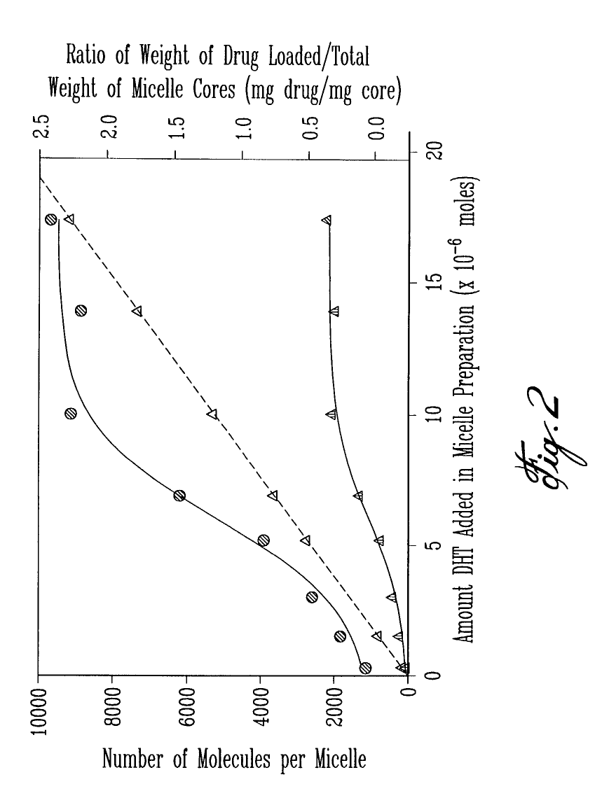

FIG. 2 includes the plot of the number of DHT molecules incorporated per micelle versus the amount of DHT added during micelle preparation. The number of molecules per micelle was found to range from a minimum of 54 to a maximum of 2,200.

The ratio of the weight of drug loaded to the weight of the core copolymer may also be calculated since the amount of drug loaded into the micelles is known. The ratio is found to increase from a minimum value of 0.10 to a maximum value of 2.4 where it levels off. The maximum value is reached when 10 μmoles or more of DHT are added during micelle preparation.

In Vitro Release Kinetic Profile of Micelle-Incorporated DHT

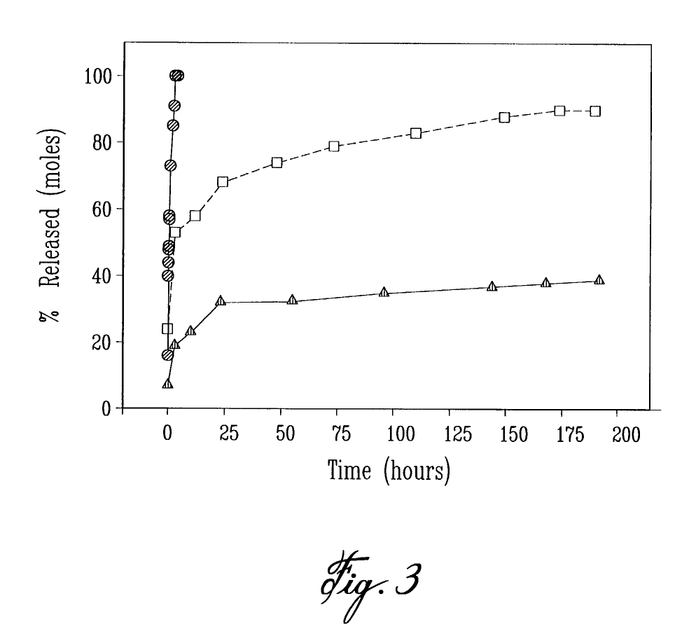

The dialysis method was used to study the release of DHT from two micelle solutions which differed in the amount of DHT that they contained. The concentration of the solutions studied were 8 mM and 40 mM. The release kinetic profiles of the two micelle-incorporated DHT solutions and the control (drug alone) over a 10-day period are plotted in FIG. 3.

As shown, the release of DHT from the 8 mM micelle-incorporated solution is much faster than that from the 40 mM solution. The release of the control solution is complete in 3 hours while 100% of the DHT from the 8 mM micelle solution is released in 8 days and 100% of the DHT from the 0.04 M solution is only released after 30 days.

In Vitro Studies

Cytotoxicity of PCL₂₀-b-PEO₄₄ Copolymer Micelles in HeLa Cell Cultures

The cytotoxicity of the empty micelles was studied in HeLa cell cultures for 24, 48 and 72 hour periods. The concentration of PCL₂₀-b-PEO₄₄ copolymer was varied from 1×10⁻⁶ to 1×10⁻³ g/g. A concentration of 1×10⁻⁴ g/g and the 24-hour period correspond to the conditions used in the transfection assays.

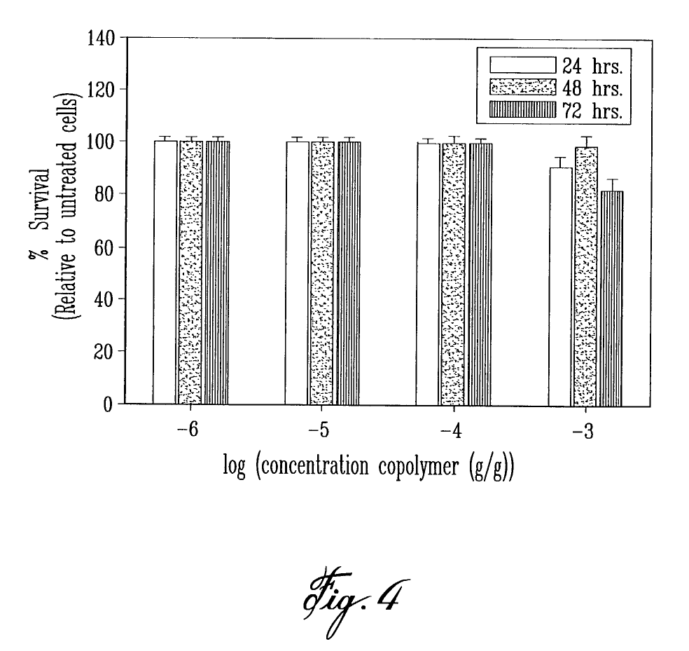

FIG. 4 shows the % of survival of cells exposed to different copolymer concentrations (1×10⁻⁶-1×10⁻⁴ g/g) for the specified time periods. The % of survival was expressed relative to the control (no copolymer added) which was taken to be 100% survival for all incubation periods (0-72 hours). The copolymer concentration of 1×10⁻³ g/g caused an insignificant degree of cell death as the percentage of cell survival fell to a low of 82% for the 72-hour incubation period.

In Vitro Biological Assay

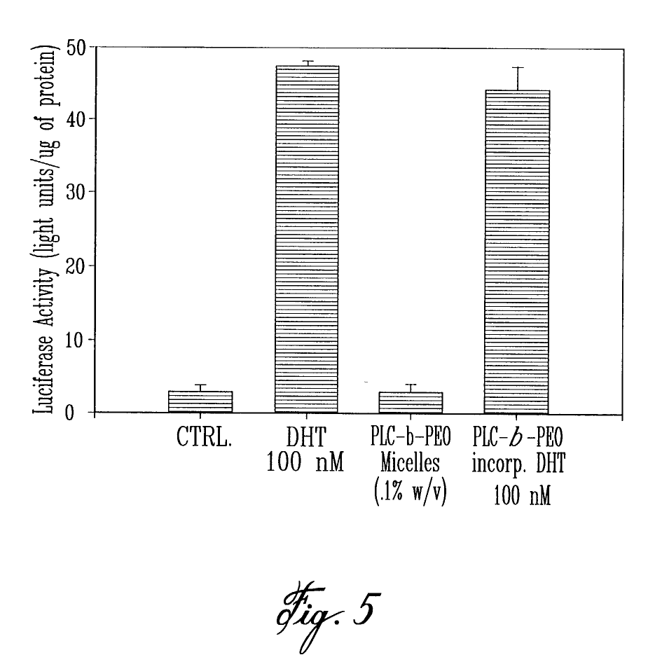

FIG. 5 shows the luciferase activity in HeLa cells cotransfected with expression vectors for MMTV-LUC and androgen receptor. The activity was determined using a highly sensitive luciferase assay system, whereby light intensity is proportional to luciferase concentration within a range of 10⁻¹⁶-10⁻¹⁸ M.

Bar 1 in FIG. 4 demonstrates the basal level of luciferase activity in cells that are untreated (negative control). As a positive control the transfected cells were treated with 100 nM DHT (bar 2), in which case the luciferase activity was 47 light units per μg of protein. As a second negative control the cells were treated with empty PCL₂₀-b-PEO₄₄ micelles, with which treatment the luciferase activity was indistinguishable from the level of activity in untreated cells. The transfected cells were also treated under identical conditions with the PCL₂₀-b-PEO₄₄ micelle-incorporated DHT such that the final concentration per well was 100 nM. There was a highly significant increase in the luciferase activity induced by both treatment with free DHT (bar 2) and micelle-incorporated DHT (bar 4). There was no significant difference between the luciferase activity induced with free drug and micelle-incorporated drug.

Discussion

Results from these studies show that the highly lipophilic model molecule DHT can be effectively incorporated into the PCL₂₀-b-PEO₄₄ copolymer non-cross-linked micelles, providing a novel delivery system for DHT with a prolonged and steady release.

The delivery system of the present invention may be used for other compounds that are highly lipophilic or toxic. The advantage of block copolymer micelles as delivery vehicles is that they may be tailor-made (e.g. size, morphology) to suit a particular application by changing the properties of the copolymer (e.g. block length, block ratio) and the conditions used in micelle preparation. The copolymer non-cross-linked micelles can also be made to be small in size (10-100 nm) and have a high degree of stability.

One major role of a delivery vehicle is to enhance the solubility of highly lipophilic drugs in an aqueous medium. The maximum amount of DHT that could be loaded into 0.1 mL of a 1% (w/w) PCL₂₀-b-PEO₄₄ micelle solution was found to be 1.3 mg (FIG. 1). This amount is equivalent to 12.8 g in 1 liter of a 1% (w/w) PCL₂₀-b-PEO₄₄ micelle solution while the solubility for DHT in water is only 42 mg/L at 25° C. The PCL₂₀-b-PEO₄₄ micelles have enhanced the solubility of DHT in water by a factor of 300.

However, the ideal non-cross-linked micelle-incorporated DHT solution will not only correspond to that which contains the maximum amount of DHT. The most useful solution of micelle-incorporated DHT would also have a high Kᵣ and a slow release profile. As mentioned previously, in a micelle solution the drug partitions between the micelles and the external medium. The ratio of the drug in the micelles to that in the external medium may be termed the relative ratio.

FIG. 1 shows that as an increasing amount of drug is added during micelle preparation, more drug is incorporated and the relative ratio increases. Yet, as the maximum loading capacity of the micelles is neared more of the drug is present in the external medium of the micelle solution so the Kᵣ decreases. The maximum value of Kᵣ is reached just prior to the point at which the loading capacity is attained (see FIG. 1). In this way it may be best to use the solution that has the highest value of Kᵣ but slightly less DHT loaded into the micelle solution.

Another factor to consider is the amount of copolymer required to deliver a specific quantity of drug. It is important that the delivery vehicle carries a sufficient quantity of drug to justify its use. The calculation of the number of drug molecules per micelle enables one to realize the extent to which the micellar delivery vehicle provides a loading space for the drug. A maximum of 2,000 molecules was found to be loaded per micelle (FIG. 2). Also, a maximum value of 2.4 was obtained for the ratio of the weight of drug loaded to the weight of the micelle cores and this demonstrates that the micelles are quite efficient carriers of DHT.

The study of the kinetic release profiles of two of the non-cross-linked micelle-incorporated DHT solutions, which differed in the amount of DHT incorporated, revealed that the more concentrated solution has the optimal release profile (FIG. 3). The release profile of the 40 mM micelle-incorporated DHT solution did not have the same initial burst release which the 8 mM solution was found to have. Also, the slow steady release from the 40 mM DHT-micelle solution was found to continue for 1 month. In this way, the 40 mM micelle-incorporated DHT solution appears to be a promising preparation to-be considered for androgen replacement therapy. However, the release kinetic profile in vitro may not be indicative of the pharmacokinetic profile in vivo. To this point, the mode of administration that has been most investigated for block copolymer micelle preparations has been by intravenous injection. However, in this instance administration by intramuscular injection may be more convenient. In vivo studies are presently underway in order to provide data on the pharmacokinetic parameters and organ biodistribution of micelle-incorporated DHT.

The study of the in vitro biocompatibility of the PCL₂₀-b-PEO₄₄ micelles in HeLa cells was required in order to ensure that this cell line could be used for the in vitro biological assay. The incubation of the PCL-b-PEO micelles for 24-72 hours in the concentration used in the transfection assay caused no noticeable cell death compared to the untreated control (FIG. 4). The in vitro biocompatibility of the micelles had been previously confirmed in PC12 cells, MCF-7 cells and primary cultures of human microglia, astrocytes and cortical neurons (Allen C., Yu Y., Maysinger D., Eisenberg A., Bioconjugate Chem. 9 (1998) 564-572).

The in vitro assay used to examine the biological activity of micelle-incorporated DHT included HeLa cells which had been cotransfected with expression vectors for MMTV-LUC and the androgen receptor. The results shown in FIG. 5 demonstrate that the preparation of micelle-incorporated DHT does not diminish the biological activity of the drug. These results also show that the drug is released from the micelle and is able to bind to the androgen receptor. Additional experiments are ongoing to decipher the mechanisms, implicated in the release of micelle-incorporated DHT and subsequent binding to the androgen receptor. The three possibilities which exist are the following: (1) the micelles remain outside the cell and the drug is released into the external medium following which it enters the cell; (2) the micelles enter the cell and release the drug into the cytoplasm after which it is transported into the nucleus; and (3) the micelles enter the nucleus where the drug is then released. Studies which employ fluorescent or radiolabeled copolymer are currently ongoing in order to determine the localization of the micelles when incubated with HeLa cells. The fact that the PCL₂₀-b-PEO₄₄ micelles are internalized into PC12 (rat pheochromocytoma) cells by an endocytotic mechanism has recently been confirmed. Similar studies must be done with HeLa cells and other cell lines, since it is known that the rate and extent of cellular internalization of small particles is cell type specific. In this way, the internalization results obtained from studies on one specific cell type cannot be assumed for the internalization into another cell type. To this point, it appears that the cellular internalization of the PCL₂₀-b-PEO₄₄ micelles into HeLa cells proceeds at a much slower rate when compared to the rate in PC12 cell cultures.

The PCL₂₀-b-PEO₄₄ copolymer non-cross-linked micelles were shown to be efficient carriers of dihydrotestosterone as they enhanced the solubility of DHT in water by 300 fold. The micelle-incorporated DHT was shown to have a slow steady release profile in PBS at 37° C. The biocompatibility of the delivery vehicle was confirmed in HeLa cells over 24-72 hour periods. Finally, the biological activity of the micelle-incorporated DHT was found to be retained as measured in HeLa cells. The results clearly demonstrate that the micelle-incorporated drug is able to reach its nuclear target.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore

What is claimed is:

1. A delivery system for a substantially sustained delivery of a steroid compound, said delivery system comprising a population of diblock copolymer non-cross-linked micelles, each said micelle defining a non-cross-linked core for containing said steroid compound.

2. A delivery system according to claim 1, wherein said diblock copolymer consists of polycaprolactone-b-poly(ethylene oxide).

3. A delivery system according to claim 2, wherein said polycaprolactone-b-poly(ethylene oxide) copolymer comprises a number of caprolactone monomers selected from 5 to 150 and a number of ethylene oxide monomers selected from 20 to 100.

4. A delivery system according to claim 3, wherein said selected number of caprolactone monomers consists of 20 and wherein said selected number of ethylene oxide monomers consists of 44.

5. A delivery system according to claim 1, wherein said steroid is a steroid hormone.

6. A delivery system according to claim 5, wherein said steroid hormone is an androgen.

7. A delivery system according to claim 6, wherein said androgen is dihydrotestosterone.

8. A method for treating a patient having a steroid level deficiency, said method comprising administering a population of diblock copolymer non-cross-linked micelles containing a steroid compound to said patient, each said micelle defining a non-cross-linked core, said micelles increasing said steroid level in said patient to a physiologically acceptable level and maintaining said level.

9. A method according to claim 8, wherein said diblock copolymer consists of polycaprolactone-b-poly(ethylene oxide).

10. A method according to claim 8, wherein said steroid level deficiency is associated with a condition selected from a group consisting of hypogonadism, microphallus, delayed puberty, post-menopause, HIV, cancer and chronic infections.

11. A method according to claim 8, wherein said steroid compound is a steroid hormone.

12. A method according to claim 11, wherein said steroid hormone is an androgen.

13. A method according to claim 12, wherein said androgen is dihydrotestosterone.
